(19) United States
(12) Patent Application Publication
(10) Pub. No.: US 2007/0167934 A1
(43) Pub. Date: Jul. 19, 2007

(54) Sheath removal hole closing device using laser welding scheme

(75) Inventors: Tsunenori Arai, Kanagawa (JP); Noriko Usami, Kanagawa (JP)

Correspondence Address: FOLEY AND LARDNER LLP;SUITE 500, 3000 K STREET NW, WASHINGTON, DC, 20007, US

(73) Assignee: KEIO UNIVERSITY, YOKOHAMA-SHI (JP)

(21) Appl. No.: 10/589,803

(22) PCT Filed: Feb. 21, 2005
(86) PCT No.: PCT/JP05/03239

§ 371 Date: Aug. 17, 2006

(30) Foreign Application Priority Data
Feb. 20, 2004 (JP) 2004-045204

(51) Int. Cl. A61B 18/18

(52) U.S. Cl. 606/2

(57) ABSTRACT

To provide a device and method for closing a sheath removal hole formed for introducing a vessel catheter by laser welding when diagnosis or treatment inside a blood vessel is performed by means of the catheter. The device for closing the sheath removal hole formed in the wall of the blood vessel by laser welding, comprising welding laser generating means, means for transmitting the welding laser, and means for monitoring the position of an end of the welding laser transmitting means, wherein the welding laser is radiated when the end of the welding laser transmitting means is located in the vessel wall.

### TECHNICAL FIELD

The present invention relates to a method and device for closing a sheath removal hole created by percutaneous transluminal angioplasty and the like using a laser welding scheme.

### BACKGROUND ART

Currently, a vessel catheter is used to perform surgery in diagnosis and treatment of diseases in a circulatory system such as a blood vessel, heart, and the like. For example, percutaneous transluminal angioplasty is performed to treat an ischemic heart disease by inserting a vessel catheter through a femoral artery into a blood vessel. When each of such vessel catheters is inserted into the blood vessel, a blood vessel into which a catheter is inserted is punctured and a sheath is kept therein to insert the catheter into the sheath (FIG. 1). After the surgery, there has been a problem of bleeding from a sheath removal hole (FIG. 2) formed when the sheath is removed. The early approach was to press the sheath removal hole area over the skin to stop bleeding, and spontaneously cure the sheath removal hole. However, this approach has taken 15 to 30 minutes to stop bleeding, and required the patient to keep quiet in bed for another several hours. Particularly when the vessel catheter is inserted through the femoral artery, a complete bed rest of not less than 12 hours has been required. Furthermore, to ensure micturition during the complete bed rest, a urethral catheter may be required to be inserted in some cases. Therefore, the early approach has heavily burdened medical staffs and has significantly reduced Quality of Life of the patient after surgery.

In contrast to the approach to rely upon spontaneous cure of the sheath removal hole, there have been developed various approaches to proactively close the sheath removal hole. These approaches include transcutaneous vascular suture and hemostasis system, in which the removal hole is sutured, and transcutaneous plaque insertion and hemostasis system, in which hemostatic plaque is inserted in the removal hole area (Hiroyoshi Yokoi, Heart View Vol. 7 No. 2 pp. 118-124 (2003)). The transcutaneous vascular suture and hemostasis system include, for example, a system known as Perclose (trademark) that requires 11 to 19 minutes to stop bleeding and 4 to 7 hours are sufficient for the bed rest after hemostasis. The success rate of the technique has been 90 to 100%. However, the technique has required experience to acquire, and because it has required a suture needle to be penetrated through the vessel wall, the penetrated needle has in some cases stuck in, resulting in surgical treatment. Therefore, it is difficult to apply the technique to a highly calcified blood vessel such as of a dialysis patient. The transcutaneous plaque insertion and hemostasis system closes the removal hole by injecting collagen gel into the sheath removal hole area, and includes VasoSeal (trademark) that requires collagen to be injected from the removal hole to the vessel wall and produces hemostasis by an acceleration effect of collagen for platelet agglutination as well as formation of collagen gel, Angio-Seal (trademark) that pinches the sheath removal hole by both injecting collagen from outside the blood vessel and inserting an anchor into the vessel, and Duett (trademark) that pinches the sheath removal hole by both injecting a mixture of collagen and thrombin from outside the blood vessel and inserting a balloon into the vessel (Johannes Brachmann et al., THE AMERICAN JOURNAL OF CARDIOLOGY VOL. 81 pp. 1502-1505 Jun. 15, 1998; Gary Gershony et al., Catheterization and Cardiovascular Interventions 45:82-88 (1998); Ulrich Gerckens et al., THE AMERICAN JOURNAL OF CARDIOLOGY VOL. 83 pp. 1658-1663 Jun. 15, 1999; Donald D. Baim et al., THE AMERICAN JOURNAL OF CARDIOLOGY VOL. 85 pp. 864-869 Apr. 1, 2000; Marie-Claude Morice et al., Catheterization and Cardiovascular Interventions 51:417-421 (2000); Michael R. Mooney et al., Catheterization and Cardiovascular Interventions 50:96-102 (2000)). In VasoSeal (trademark), hemostasis time is several minutes, and only requires about 5 hours for the bed rest. The success rate of the technique is 88 to 100%. However, it has suffered from a risk of complications such as infection and allergic reaction due to insertion of collagen. In addition, it could not be applied to a thin patient whose skin and blood vessel are close to each other. In Angio-Seal (trademark), hemostasis time has been 2 to 4 minutes, bed rest time has been 6 to 8 hours, and the success rate of the technique has been 91 to 100%. Similarly to VasoSeal (trademark), however, it has suffered from a risk of infection and allergic reaction due to insertion of collagen. In addition, because it requires an anchor to be inserted into the vessel, it has been difficult to apply the technique to a site having a large fatty deposit in the removal hole area. In Duett (trademark), hemostasis time has been 4 to 6 minutes, bed rest time has been 2 to 6 hours, and the success rate of the technique has been 98 to 100%. Similarly to VasoSeal (trademark) and others, however, it has suffered from a risk of infection and allergic reaction due to insertion of collagen. In addition, because it requires a balloon to be inserted into the vessel, it has been incompatible with a blood vessel of less than 6 mm in its inner diameter, and has been incompatible with, for example, a thin patient whose skin and blood vessel are close to each other. Furthermore, the transcutaneous plaque insertion and hemostasis system requires experience for accurately injecting collagen into the sheath removal hole area.

As described above, conventional sheath removal hole closing schemes have suffered from various problems, and there is a need for a sheath removal hole closing scheme that rapidly produces hemostasis, allows a patient to early leave his/her sickbed and/or hospital, and improves Quality of Life, without a risk of complications.

On the other hand, various researches have conventionally been conducted to weld living tissues using a laser (Hasegawa et al., Lasers in Surgery & Medicine 29(1): 62-9, 2001; Tang J, et al., Lasers in Surgery & Medicine 22(4): 207-11, 1998; Tang J. et al., Lasers in Surgery & Medicine 21(5): 438-43, 1997; Seaman EK. Et al., Journal of Urology 158(2): 642-5, 1997; Menovsky T. et al., Lasers in Surgery & Medicine 19(2): 152-8, 1996; Bass T. S. et al., Lasers in Surgery & Medicine 12(5): 500-5, 1992; White R A. Et al., Lasers in Surgery & Medicine 8(1):83-9, 1988). In these approaches, an argon laser, a semiconductor laser, a carbon dioxide laser, and the like are used for a laser beam, and indocyanine green (ICG), fluorescein, iron oxide, and the like are used as a pigment for absorbing laser energy. The welding mechanisms soften collagen at approximately 60° C. and entangle collagen fibers. Although there has been reported an application to a blood vessel among these laser welding schemes (JP Patent Publication (Kokai) No. 2001-190566A), it is directed to a coronary artery, and its object is to provide anastomasis that bonds a broken vessel wall.

Patent Document 1: JP Patent Publication (Kokai) No. 2001-190566A

### DISCLOSURE OF THE INVENTION

It is an object of the present invention to provide a device and method for closing a sheath removal hole formed for introducing a vessel catheter by laser welding when diagnosis or treatment inside a blood vessel is performed by means of the vessel catheter.

The present inventors have intensively examined the possibility of developing a sheath removal hole closing scheme using laser welding. As a result, the inventors have found that the blood vessel can be welded in its sheath removal hole area to close the sheath removal hole by removing the catheter after using the vessel catheter for surgery, inserting into the sheath a fiber capable of radiating a welding laser onto the sheath disposed in the vessel wall, and radiating the laser onto the sheath removal hole site while the sheath is being removed (FIG. 3). At this time, only the vessel wall site must be irradiated by the laser, and it is required to monitor the location of the end of the fiber radiating the laser. Therefore, weak monitoring light is radiated from the fiber end and backscattered light is detected so that it can be determined where the fiber end is located; in blood, in the vessel wall, or in a surrounding tissue. This allows the vessel wall to be locally irradiated by the welding laser, and allows the sheath removal hole to be reliably closed without damaging other tissues.

The present invention is as follows:

[1] a device for closing a sheath removal hole formed in the wall of a blood vessel by laser welding, comprising welding laser generating means, means for transmitting the welding laser, and means for monitoring the position of an end of the welding laser transmitting means, wherein the welding laser is radiated when the end of the welding laser transmitting means is located in the vessel wall;

[2] the device for closing a sheath removal hole in [1], wherein the welding laser is a laser capable of heating the vessel wall;

[3] the device for closing a sheath removal hole in [2], wherein the welding laser is a continuous laser capable of heating the vessel wall;

[4] the device for closing a sheath removal hole in [3], wherein the welding laser is selected from the group consisting of a semiconductor laser, a Nd:YAG laser, and a second harmonics of a Nd:YAG laser;

[5] the device for closing a sheath removal hole in any of [1] to [4], wherein the means for monitoring the position of an end of the welding laser transmitting means includes means for generating monitoring light, means for transmitting the monitoring light, and means for detecting backscattered light of the monitoring light, and wherein an end of the monitoring light transmitting means and the end of the welding laser transmitting means are located at the same position, the monitoring light that is light having a wavelength absorbable by substances present in blood is radiated, the backscattered light of the radiated monitoring light is detected, and the position of the end of the welding laser transmitting means is determined based on the intensity of the detected light:

[6] the device for closing a sheath removal hole in [5], wherein in the means for monitoring the position of an end of the welding laser transmitting means, the monitoring light is light having a wavelength absorbable by hemoglobin, and wherein it can be determined where the end of the welding laser transmitting means is located; in blood, in the vessel wall, or in a surrounding tissue of the blood vessel;

[7] the device for closing a sheath removal hole in [6], the light having a wavelength absorbable by hemoglobin used to monitor the position of an end of the welding laser transmitting means is selected from the group consisting of a semiconductor laser having a wavelength of 810 nm, a He—Ne laser having a wavelength of 543 nm, and a second harmonics of Nd:YAG laser having a wavelength of 532 nm;

[8] the device for closing a sheath removal hole in any of [1] to [7], wherein the welding laser transmitting means and the monitoring light transmitting means are a common flexible transmitting means;

[9] the device for closing a sheath removal hole in [8], wherein the flexible transmitting means is selected from the group consisting of a quartz glass fiber, a plastic fiber, and a hollow medical waveguide;

[10] the device for closing a sheath removal hole in any of [1] to [9], wherein the welding laser generating means and the monitoring light generating means are a common semiconductor laser or Nd:YAG laser second harmonics generator;

[11] the device for closing a sheath removal hole in any of [1] to [10], further comprising a means for supplying a welding laser energy absorbing pigment to the sheath removal hole;

[12] the device for closing a sheath removal hole in [11], wherein the welding laser energy absorbing pigment is indocyanine green;

[13] a control method for determining the position of an end of a light transmitting fiber and radiating a welding laser through the light transmitting fiber onto the wall of a blood vessel in which a sheath removal hole is formed, in order to close the sheath removal hole by laser welding, comprising the steps of:

(a) radiating weak light used for determining a surrounding tissue onto the light transmitting fiber inserted in the sheath inserted in the blood vessel, the fiber connected with a light generator;

(b) measuring backscattered light of the radiated weak light by a detector;

(c) determining a tissue surrounding the end of the light transmitting fiber; and

(d) radiating welding laser if it is determined that the tissue surrounding the end of the light transmitting fiber is the vessel wall;

[14] a device for monitoring the position of an end of monitoring light transmitting means, comprising means for generating monitoring light, means for transmitting the monitoring light, and means for detecting backscattered light of the monitoring light, wherein the monitoring light that is light having a wavelength absorbable by substances present in blood is radiated, the backscattered light of the radiated monitoring light is detected, and the position of the end of the monitoring light transmitting means is determined based on the intensity of the detected light;

[15] the device for monitoring the position of an end of a monitoring light transmitting means in [14], wherein in means for monitoring the position of an end of the monitoring light transmitting means, the monitoring light is light having a wavelength absorbable by hemoglobin, and wherein it can be determined where the end of the monitoring light transmitting means is located; in blood, in the vessel wall, or in a surrounding tissue of the blood vessel; and

[16] the device for monitoring the position of an end of a monitoring light transmitting means in [14] or [15], the light having a wavelength absorbable by hemoglobin used to monitor the position of an end of the monitoring light transmitting means is selected from the group consisting of a semiconductor laser having a wavelength of 810 nm, a He—Ne laser having a wavelength of 543 nm, and a second harmonics of Nd:YAG laser having a wavelength of 532 nm.

The specification incorporates the content of Japanese Patent Application No. 2004-045204 and/or drawings thereof, which is the basis of priority for the present application.

### BRIEF DESCRIPTION OF THE DRAWINGS

FIG. 1 shows a method for diagnosis and treatment of a blood vessel using a vessel catheter;

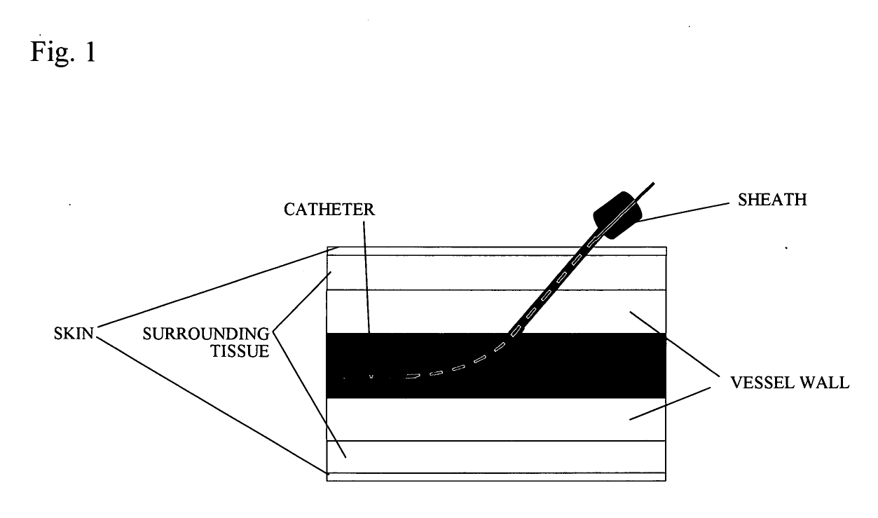

FIG. 2 is a photograph showing a sheath removal hole;

FIG. 3 is a schematic view of a sheath removal hole closing scheme using a laser welding scheme;

FIG. 4A shows a method for determining a tissue utilizing backscattered light;

FIG. 4B shows a trail and the intensity of light in the method of FIG. 4A, in which the thickness of the arrow represents the intensity of light, the left shows a case of small absorption and the right shows a case of large absorption;

FIG. 5 shows a theoretical variation in backscattered light in tissues (in blood, in the vessel wall, and in a surrounding tissue);

FIG. 6A is a schematic view of an experiment of sheath removal hole closing using the laser welding scheme, showing a front view of an experimental setup;

FIG. 6B is a schematic view of an experiment of sheath removal hole closing using the laser welding scheme, showing a side view of an experimental setup;

FIG. 7 is a photograph showing a cross section of a sheath removal hole welded and closed by the laser welding scheme;

FIG. 8 is photographs of a stained cross section of a sheath removal hole welded and closed by the laser welding scheme, in which a blue portion represents collagen fibers, a pale red portion represents elastin fibers, a blackish brown portion represents cell nuclei, and the right photograph is a magnification of the left photograph in the rectangle area;

FIG. 9 is a schematic view of an experiment of backscattered light measuring, where the laser used is a He—Ne laser (green) having a wavelength of 543 nm and an output power of 1 mW, the laser passes through a beam splitter and a lens to reach a sample through a fiber having a core diameter of 400 μm and NA 0.25, and the light returned from the sample passes rough the fiber, lens and beam splitter, and is recognized at a photodiode;

FIG. 10 shows a blood vessel model used in the experiment of backscattered light measuring, where the aorta is used to simulate a femoral artery, and the cardiac muscle is used to simulate a surrounding tissue;

FIG. 11A shows measurements of backscattered light;

FIG. 11B shows materials used in measuring backscattered light;

FIG. 12 is a diagram of a sheath removal hole closing device that utilizes laser welding; and

FIG. 13 shows an intravascular lumen pressurizing device.

### DESCRIPTION OF SYMBOLS

1 LIGHT GENERATOR
2 FIBER
3 BEAM SPLITTER
4 LENS
5 FILTER
6 PHOTODETECTOR
7 SHEATH
8 VESSEL WALL
9 BLOOD VESSEL (BLOOD)
10 SURROUNDING TISSUE
11 LASER BEAM

### Best Mode for Carrying Out the Invention

The present invention will now be described in detail.

The device according to the present invention may be used to close a sheath removal hole formed in the wall of a blood vessel after diagnosis or treatment when removing a sheath, which is inserted to introduce a vessel catheter when the catheter is inserted into a blood vessel for the diagnosis or treatment in the blood vessel. The intended blood vessel is not limited provided that a vessel catheter can be inserted in the blood vessel, and may include a femoral artery, a stapedial artery, and the like.

There are various diameters for sheaths that are usually used, and those having a size of 5 F (French) to 11 F are used depending on the type and size of a blood vessel to which the sheath is inserted. The device according to the invention used to close a sheath removal hole may be applied to any size of sheath removal hole.

1. Arrangement of the Device Used to Close a Sheath Removal Hole

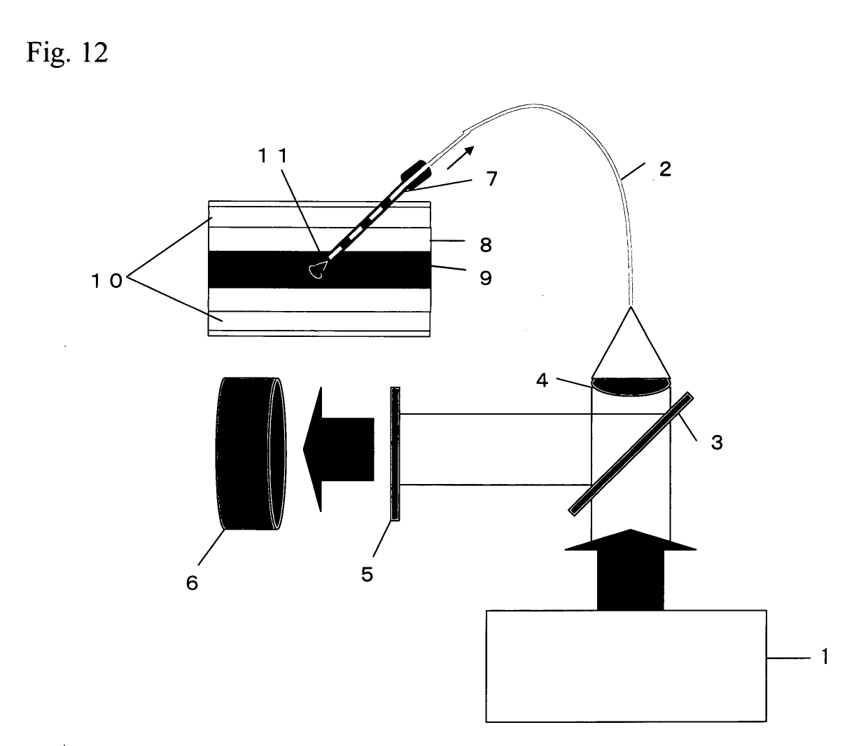

The device according to the invention used to close a sheath removal hole includes, at least, welding laser generating means, means for transmitting the welding laser to the vessel wall, and means for monitoring the position of an end of the welding laser transmitting means. An exemplary arrangement of the device according to the invention is shown in FIG. 12. The device according to the invention, however, is not intended to be limited to the device arrangement shown in FIG. 12.

(1) Welding Laser Generating Means

The welding laser generating means (laser source) may be a conventional therapeutic near-infrared laser generator, and in laser welding by means of the device according to the invention, the vessel wall having a sheath removal hole is irradiated by the laser, and locally heated to soften collagen in the vessel wall for welding. The temperature generated by the heating is 60° C. to 70° C.

The laser seed may be a laser capable of heating a vessel wall, preferably a continuous laser capable of heating a vessel wall. The wavelength range preferably allows for reasonable permeability to the vessel wall, and the permeability in this case preferably allows for 50 μm to 1 cm in permeable length of light. Specifically, the laser includes those having a wavelength of 300 nm to 2.5 μm, or of 4 μm to 11 μm, and may be a laser having a wavelength transmittable by flexible transmitting means such as a quartz glass fiber, a plastic fiber, and a hollow medical waveguide. For such a laser, for example, a semiconductor laser (810 nm), a Nd:YAG laser (1064 nm), a Nd:YAG second harmonics having a wavelength of 532 nm, and the like are used.

In addition, a pigment that absorbs laser energy may be supplied to the sheath removal hole area to stain the area when laser welding is performed. After the sheath removal hole is stained, it may be locally irradiated by a laser so that the hole is welded. As a pigment for absorbing laser energy, a pigment that highly absorbs a laser wavelength which is highly permeable to a blood vessel and can be administered to a living body is selected, and for example, indocyanine green and iron formulation such as iron oxide are used. The iron oxide includes saccharated iron oxide like Fesin (registered trademark, Yoshitomiyakuhin Corporation). A combination of indocyanine green and semiconductor laser, or a combination of the iron formulation and Nd:YAG laser is preferable as a combination that can generate a temperature of 60° C. to 70° C. locally at a sheath removal hole area. However, the invention is not intended to be limited to these combinations, and any combination of laser seed and pigment may be used, provided that it satisfies the conditions of laser seed and pigment as described above and, when combined, it can generate a high temperature of 60° C. to 70° C. locally at a sheath removal hole area.

The laser generator includes, for example, a semiconductor laser generator UDL-60 (Olympus Corporation) and the like.

Local temperature rise depends on the laser intensity and duration of exposure, and excessively high intensity and short pulses cause disturbances due to sound waves in tissues. Therefore, it may be advantageous that the duration of radiation of laser is relatively long pulses or continuous. On the other hand, however, excessively long duration of exposure causes damages on surrounding tissues, and therefore, a procedure using a continuous laser in a relatively short duration will be required. A preferred duration of exposure is 1 ms to 10 seconds, and the shorter within this range, the more preferable it is for avoiding surrounding damages. However, because welding can be considered as a certain chemical reaction process, a certain length of duration of exposure will be required depending on a welding temperature. With this point taken into consideration, a preferred duration of exposure is 5 ms to 10 seconds, and more preferably 4 to 10 seconds. The duration of exposure can be selected as appropriate within the range described above depending on collagen content in the sheath removal hole area, size of the sheath removal hole area, and the like. Radiations in the duration described above may be repeated from the start to the end of radiation (intermittent radiation).

The output power of the laser used is 0.05 to 30 W/mm². To satisfy the short time radiation conditions described above, an output power as large as possible within this range is preferable.

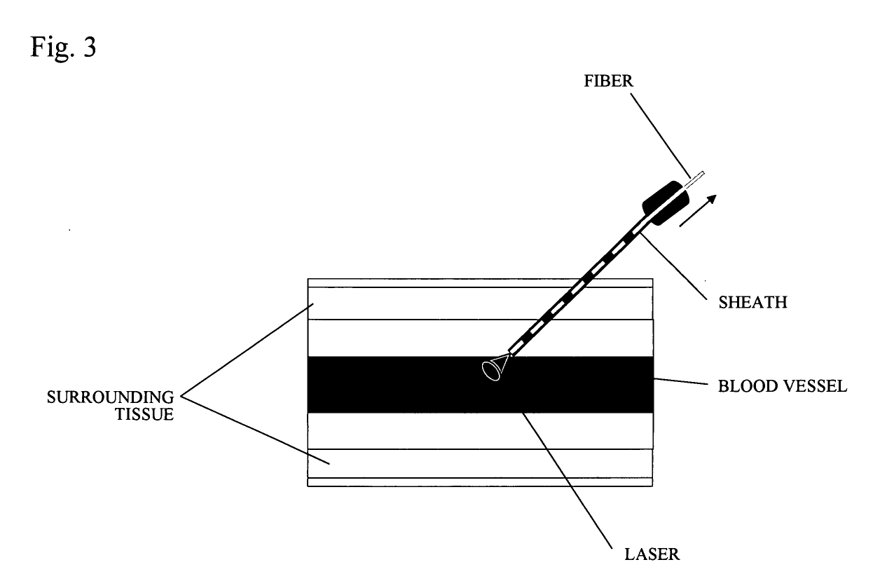

Furthermore, in order to weld and close a sheath removal hole, the sheath removal hole must be pressed with a suitable pressure while the laser is radiated. The sheath is typically inserted into a blood vessel at an angle of 45 degrees. Therefore, the sheath removal hole is also formed at an angle of 45 degrees relative to the vessel wall (FIG. 3). In this case, the sheath removal hole is pressed by blood pressure produced by blood flown in the blood vessel, and the sheath removal hole is closed by itself The laser may be radiated onto the closed sheath removal hole. However, depending on the angle of formed sheath removal hole, size of the sheath removal hole, and/or the blood pressure, the blood pressure in the blood vessel alone is not enough to close the sheath removal hole. In such a case, it is required to apply pressure to the sheath removal hole area to close sheath removal hole by, for example, holding down the sheath removal hole area from outside the blood vessel. A balloon or stent may be used to apply pressure from inside the blood vessel. Applied pressure at this time is 0.05 to 1 kg/cm², preferably 0.1 to 1 kg/cm², and more preferably on the order of 130 g/cm², which corresponds to the artery pressure.

Therefore, a preferred aspect of the present invention for closing a sheath removal hole by laser welding is to use a semiconductor laser for the laser seed and indocyanine green for the pigment, or to use Nd:YAG laser for the laser seed and iron oxide formulation for the pigment, in such a way that the sheath removal hole area is locally irradiated by a continuous laser for 1 ms to 10 seconds to generate a high temperature of 60 to 70° C., causing collagen to be softened and entangled to weld and close the sheath removal hole.

(2) Means for Transmitting a Welding Laser

Means for transmitting the welding laser to the vessel wall include flexible transmitting means that can transmit a laser from a laser generator to a sheath removal hole. The flexible transmitting means include a quartz glass fiber, a plastic fiber, a hollow medical waveguide, and the like. Such flexible transmitting means may be herein referred to as an optical fiber or a fiber. The laser is transmitted in the fiber and radiated from an end of the fiber.

The fiber is housed in a suitable protective tube, for example, a sheath or a catheter inserted in the sheath, and connected with the laser generator at the other end. The end of the fiber may be provided with a suitable laser beam radiating device such as a lens. The fiber used in the present invention may be any fiber having a wide variety of diameters ranging from a highly thin fiber of on the order of 0.05 to 0.6 mm in diameter to that having visible thickness.

(3) Means for Monitoring the Position of an End of the Welding Laser Transmitting Means

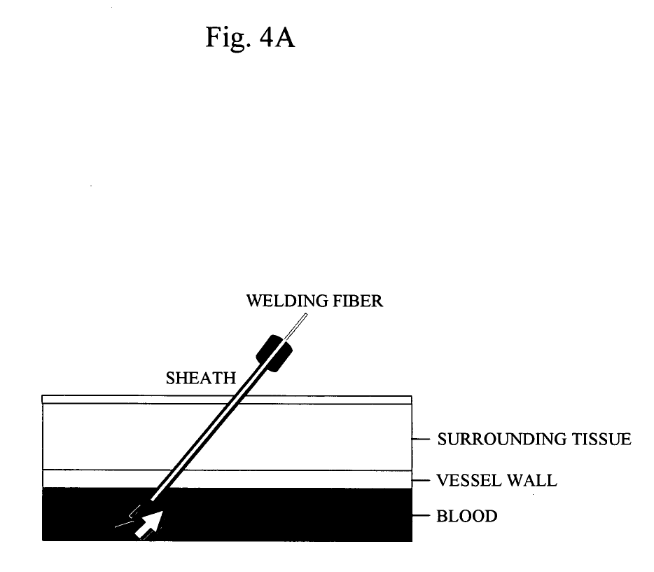

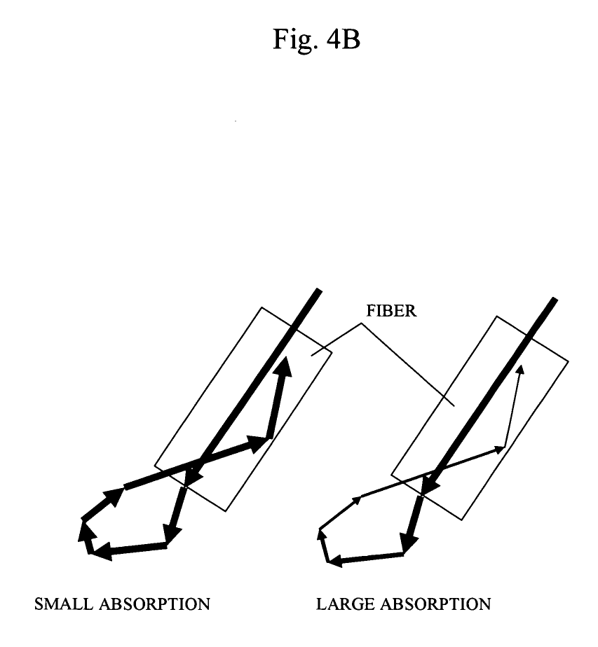

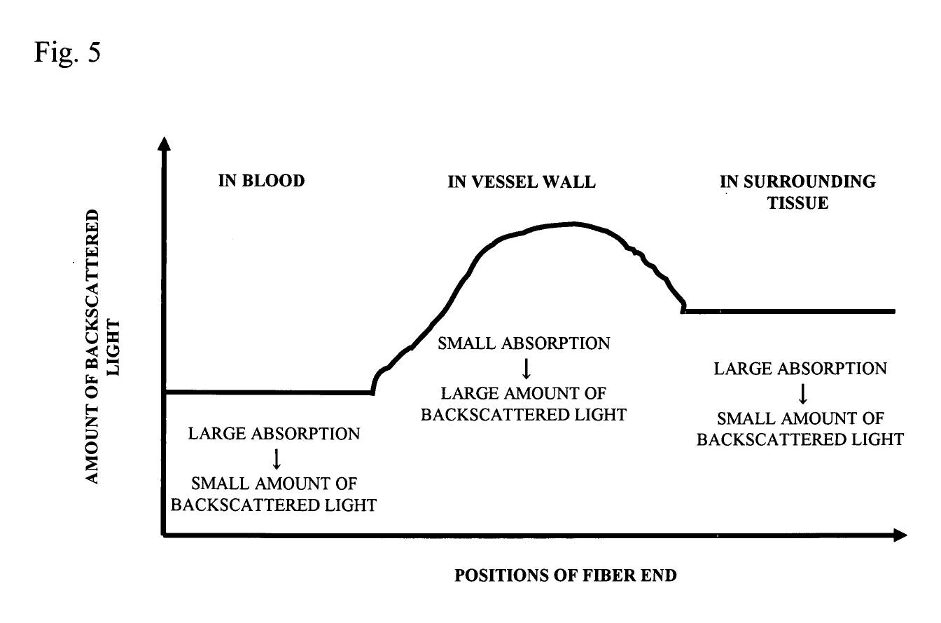

When a sheath removal hole is to be irradiated by a welding laser and the welding laser transmitting fiber is moved along the sheath removal hole, the welding laser radiating portion located at the end of the welding laser transmitting means may be present anywhere in a blood vessel, in the vessel wall, or in a surrounding tissue outside the blood vessel (FIG. 3). When the sheath removal hole is to be closed by means of a device according to the invention, only the vessel wall having the sheath removal hole formed must be irradiated by the welding laser. Therefore, the location of the end of the fiber from which the welding laser is radiated is monitored, so that the welding laser is radiated only when the end of the fiber is in the vessel wall. In this case, it is sufficient to determine which tissues surrounds the end of the fiber that transmits and radiates the welding laser. The determination of a tissue may be accomplished by using a fact that a specific substance in the tissue absorbs a specific wavelength of light: monitoring light having a wavelength absorbed by a substance that is present in a small amount within the vessel wall and present in a large amount within blood and surrounding tissues may be radiated from the position of the end of the welding laser transmitting fiber to detect backscattered light of the light. The backscattered light here refers to light that returns to the fiber again after the light that is radiated from the fiber is absorbed and scattered in tissues near the radiating portion. FIGS. 4A and B show schematics of a method for monitoring the position of the end of the monitoring light transmitting means. The black arrow in FIG. 4A represents monitoring light that is radiated from the end of the fiber, and the white arrow represents the backscattered light. FIG. 4B shows light that is radiated from the fiber and scattered before it returns to the fiber as backscattered light, and the thick arrow represents strong light, and the thin arrow represents weak light. As shown in the figures, when tissues surrounding the end of the fiber absorb a large amount of light, the returned backscattered light is weak, and when tissues surrounding the end of the fiber absorb a small amount of light, the returned backscattered light is strong. A substance that is present in a small amount within the vessel wall and in a large amount within blood and surrounding tissues includes substances contained within blood, and hemoglobin is particularly preferable. Hemoglobin is chromoprotein, and absorbs a specific wavelength of light. Therefore, light absorption and scattering characteristics vary depending on hemoglobin content of each tissue, and therefore it is possible to determine a tissue of the site irradiated by light, by detecting the backscattered light. Theoretically, because the inside of a blood vessel is filled with blood, resulting in high hemoglobin content and large absorption, the amount of backscattered light is small. The vessel wall contains little hemoglobin (although blood enters the sheath removal hole), and this means small absorption and a large amount of backscattered light. Surrounding tissues outside the vessel wall (for example, muscle tissue) include capillary vessels and the like, resulting in relatively high hemoglobin content and relatively large absorption: therefore, the amount of backscattered light is relatively small. FIG. 5 shows a variation in backscattered light in tissues predicted from a theory. In FIG. 5, the axis of abscissas shows positions of the end of the fiber that radiates monitoring light, and the axis of ordinates shows the amount of backscattered light.

The monitoring light may be light having a wavelength of 200 nm to 900 nm. The peak wavelengths of light absorbed by hemoglobin are at approximately 400 and 550 nm. However, even deviated from the peaks, the light can be absorbed by chromoprotein, or hemoglobin, and therefore it may be used as monitoring light in a device according to the invention. For example, even though the wavelength of welding laser is deviated from the peak wavelengths of absorption of hemoglobin, the laser may also be used as monitoring light. In addition, the light intensity may be small, and weak light having an output power of 0.01 mW to 1 mW may be used. Particularly when the welding laser is also used as monitoring light, the output power must be reduced to obtain weak light while it is used as monitoring light in order to avoid influence on tissues. The monitoring light includes, for example, He—Ne laser (green) having a wavelength of 543 nm and an output power of 1 mW. The monitoring light is generated in an external light generator, transmitted through a monitoring light transmitting fiber, and radiated from an end of the fiber. The fiber used at this time may be a fiber having the same diameter as the welding laser transmitting fiber. The backscattered light reenters the transmitting fiber that radiates the monitoring light, and comes back through the fiber. To detect the backscattered light, a detector for monitoring the backscattered light may be connected to the fiber that the backscattered light enters and comes back through. A beam splitter may be provided in the way of the fiber to alter the course of light returning through the optical fiber, and further the light may be caused to pass through a suitable bandpass filter to select only a desired wavelength of light before directing to the scattering light detector. The scattering light detector may be anything that can detect light without limitation, and for example, a silicon photodiode may be used. At this time, the intensity of backscattered light abruptly varies when the end of the monitoring light transmitting fiber moves from within blood into the vessel wall and when it moves from the vessel wall into a surrounding tissue (FIG. 5), and therefore, a variation of the backscattered light may be monitored.

The monitoring light transmitting fiber may be provided separately from the welding laser transmitting fiber. In this case, however, the end of the monitoring light transmitting fiber and the end of the welding laser transmitting fiber must be aligned. On the other hand, a single fiber may be used for both transmitting the welding laser and transmitting the monitoring light. Using a single fiber to transmit both of light is preferable in that a portion inserted to a blood vessel through a sheath of the device according to the invention may be made thin.

When the welding laser transmitting fiber and the monitoring light transmitting fiber are integrated, the welding laser generating means and the monitoring light generating means may be connected at one end of the fiber, and the light sources may be switched as appropriate. As described above, because the welding laser may also be used as the monitoring light by changing the intensity of the light, a laser generator such as a semiconductor laser generator may be connected and high intensity light may be radiated when laser welding is performed, and weak light may be radiated when the position of the end of fiber is monitored.

(4) Other Means

As necessary, temperature measuring means such as thermocouple may be provided at the end of the fiber so that a temperature variation can be measured in a portion irradiated by the welding laser. The temperature rise that can be monitored by the temperature measuring means may be used as an indicator to determine to what extent the sheath removal hole is welded and closed.

Further, the device according to the invention may include means for supplying a pigment for increasing the welding efficiency of the welding laser. The means for supplying a pigment to the sheath removal hole is means for supplying a pigment absorptive of laser energy, including indocyanine green and iron oxide such as Fesin. When such means is provided in the device, a liquid delivering tube is provided in a tube such as catheter that houses the light transmitting fiber. Providing means for injecting pigment solution near the end of the tube allows the pigment to be supplied to the sheath removal hole. The pigment solution may, for example, be delivered by means of a pump such as a syringe, a peristaltic pump, and the like. The pigment solution may be injected, for example, through a small hole or a slit-shaped hole provided on the end of the liquid delivering tube. It is desirable that the dye concentration in this case be sufficiently lower than the tolerance. The amount and concentration of the supplied pigment may be changed as appropriate either when it is supplied by intravenous administration or when it is supplied by means of the pigment supplying means. For example, when the pigment is directly supplied by means of the pigment supplying means to the sheath removal hole, it may be sufficient to supply the pigment having a concentration of several μg to several tens mg/mL in an appropriate amount. However, some pigments may have an adverse effect on the human body, and therefore a dose may be determined with LD50 values and the like taken into consideration for each pigment. Even without a dedicated means provided in the device, the pigment may be supplied by administering the pigment to the sheath removal hole area of the patient before treatment by means of the treatment device according to the invention. For example, the pigment solution may be injected through an appropriate tube or injector into an area where the sheath is inserted before the sheath is removed. The pigment may be supplied anytime before the welding laser is radiated, and a good time may be before the welding laser radiating fiber is inserted or may be immediately before the laser is radiated after the welding laser radiating fiber is inserted.

In addition, when the device according to the invention is used to close the sheath removal hole by laser welding, the end of the fiber is moved by 0.1 mm or less in order to determine the position of the end of the fiber by measuring backscattered light. Although the movement may be manually performed, suitable precision propelling means may be provided to accomplish the movement. The precision propelling means includes, for example, an application of a micrometer screw and the like.

The means for monitoring the position of an end of the welding laser transmitting means included in the sheath removal hole closing device according to the invention may be used as a device for monitoring the position of the end of the monitoring light transmitting means, and it may be used to diagnose the conditions in the blood vessel by radiating various lasers, or may be combined with a diagnosis/treatment device using a vessel catheter that treats diseases in the blood vessel, to monitor the sites in the blood vessel to be diagnosed or treated.

2. Usage of a Device According to the Invention

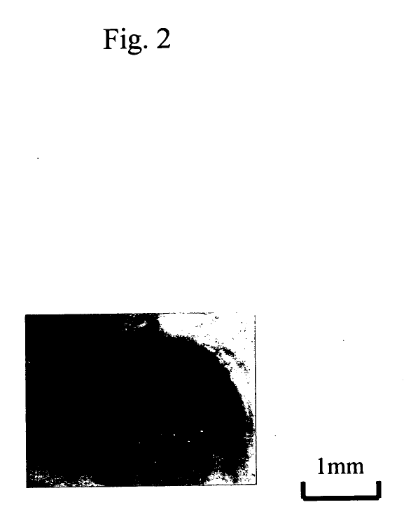

FIG. 2 shows a condition of a sheath removal hole. FIG. 12 shows an arrangement of the device according to the invention for closing the sheath removal hole by laser welding. In FIG. 12, a laser generator can radiate a welding laser and a monitoring light (laser), and a light transmitting fiber can transmit both the welding laser and the monitoring light (laser).

The usage of the device according to the invention will be described based on FIG. 12. A fiber portion 2 of the device according to the invention may simply be inserted through a sheath 7 that is inserted into the blood vessel for inserting a vessel catheter, so that an end of the fiber 2 reaches the sheath removal hole. Because the position of the end of the fiber 2 cannot be known only by inserting the fiber 2, the light generator (laser generator) 1 in FIG. 12 is caused to generate weak monitoring light, and the light is transmitted through the fiber 2 and radiated from the end of the fiber 2. The monitoring light is absorbed and scattered on a tissue at the irradiated area, and the scattered light reenters the fiber 2 as backscattered light and comes back. The course of the returned light is altered by a beam splitter 3 and directed to a photodetector (silicon photodiode) 6 through a suitable filter 5 to measure the intensity of the light. At this time, the backscattered light of the radiated weak light is measured while the position of the end of the fiber 2 is being shifted. The location of the end of the fiber 2 can be known by means of the varying backscattered light. Therefore, while the position of the end of the fiber 2 is being moved, the monitoring light is radiated and the intensity of the backscattered light is measured to monitor the intensity variation. When the end of the fiber 2 is moved between blood and the vessel wall or between the vessel wall and a surrounding tissue, the variation of the intensity abruptly varies as shown in FIG. 5, so that the end of the fiber 2 can be located.

Once it is confirmed that the end of the fiber 2 is in the blood in this way, the weak monitoring light generated in the light generator 1 is radiated while the fiber 2 is being slowly pulled out. In FIG. 12, an arrow along a portion of the sheath 7 shows a direction in which the position of the end of the fiber 2 is shifted. The backscattered light returning through the fiber 2 is monitored, and at the time when it is determined that the intensity of the backscattered light rises and the end of the fiber 2 is moved into the blood vessel, the light generator 1 is caused to generate a welding laser, which is transmitted through the fiber 2 and radiated onto the sheath removal hole from the end thereof Radiating the weak light, measuring the backscattered light, locating the end of the fiber 2, and moving the position of the end of the fiber 2 are repeated, so that the sheath removal hole may be welded and closed by means of welding laser while the position of the end of the fiber 2 is being moved. Instead of radiating the welding laser while moving, the laser may be radiated at a suitable point or multiple points in the sheath removal hole. The points in the sheath removal hole to be irradiated include a point where the end of the fiber 2 moves from within blood 9 into the vessel wall 8, a point immediately before the end of the fiber 2 moves from within the vessel wall 8 into a surrounding tissue 10, and any points between these two points. For the point immediately before the end of the fiber 2 moves from within the vessel wall 8 into a surrounding tissue 10, the fiber 2 may be slightly pushed in once it is confirmed that the end of the fiber 2 moves from within the vessel wall 8 into the surrounding tissue 10 by monitoring the backscattered light.

Contrary, once it is confirmed that the position of the fiber 2 is within the surrounding tissue 10, the welding as described above may be performed while the fiber 2 is being pushed in.

When the welding laser is radiated onto the sheath removal hole, the sheath inserted in the vessel wall needs to be pulled out, and it may be pulled along with the fiber. For example, at the time when it is confirmed that the position of the end of the fiber is in blood, the fiber and the sheath may be immovably fastened together and the sheath may be pulled out so that the sheath and the fiber is pulled out at the same time.

3. Method for Controlling the Radiating Position of a Welding Laser in a Sheath Removal Hole Closing Scheme Using Laser Welding

The present invention encompasses a control method for determining the position of a sheath removal hole and radiating a welding laser in order to close the sheath removal hole by laser welding.

This is a method for controlling the radiating position of the welding laser, in which weak light is radiated onto the sheath removal hole and backscattered light of the weak light is monitored in order to determine whether the area irradiated by the weak light is located in blood, in the vessel wall, or in a surrounding tissue of the blood vessel, and if it is determined that the irradiated area is in the vessel wall, a welding laser for closing the sheath removal hole is radiated.

The control method includes the following steps:

radiating weak light used for determining a surrounding tissue onto the monitoring light transmitting fiber inserted in the sheath inserted in the blood vessel, the fiber connected with a monitoring light generator;

measuring backscattered light of the radiated weak light by a detector;

determining a tissue surrounding the end of a welding laser transmitting fiber located at the same position as the end of the monitoring light transmitting fiber; and

radiating welding laser if it is determined that the tissue surrounding the end of the welding laser transmitting fiber is the vessel wall.

When the welding laser transmitting fiber and the monitoring light transmitting fiber are a common fiber, the method includes the following steps:

radiating weak light used for determining a surrounding tissue onto the light transmitting fiber inserted in the sheath inserted in the blood vessel, the fiber connected with a light generator;

measuring backscattered light of the radiated weak light by a detector;

determining a tissue surrounding the end of the light transmitting fiber; and

radiating welding laser if it is determined that the tissue surrounding the end of the light transmitting fiber is the vessel wall.

The present invention will be specifically described with reference to the following examples. However, the invention is not intended to be limited to these examples.

### EXAMPLE 1

Laser Welding

A sheath removal hole model was constructed and a device according to the invention was used to accomplish sheath removal hole closing.

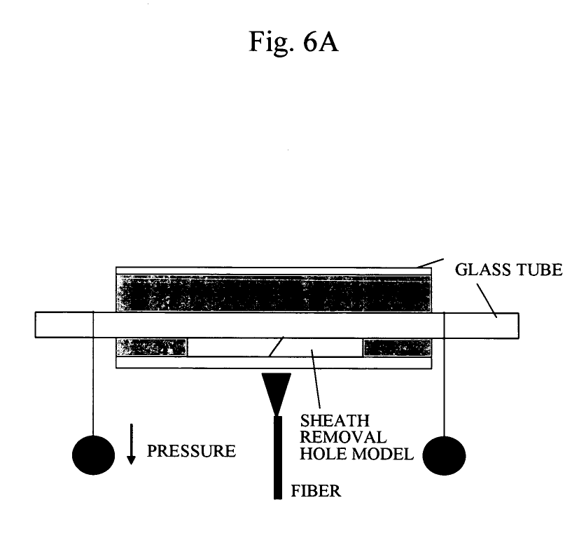

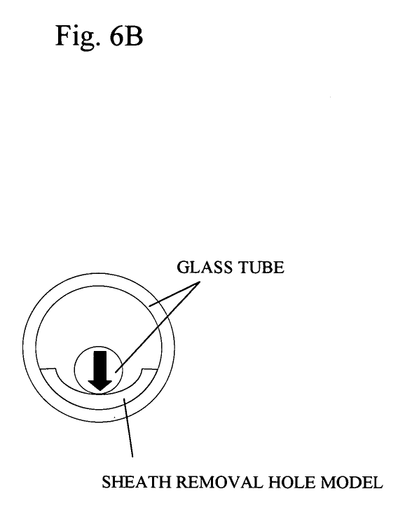

An extracted carotid artery of a pig (2 cm long and 0.5 cm wide in a blood flow direction) was punctured with a 4F sheath at an angle of 45 degrees and the sheath was kept for an hour and removed to form an sheath removal hole, which was used as the sheath removal hole model. Indocyanine green (absorption peak wavelength at 805 nm) in an amount of 2.5 mg/mL was dispensed and added to the sheath removal hole using a syringe. As shown in FIG. 6, the sheath removal hole model was placed within a hollow glass tube having an inner diameter of 9.4 mm in close contact with the inner periphery of the tube. In addition, a glass rod having a diameter of 5 mm was placed on the sheath removal hole model, and weights of 130 g was strung near both ends of the glass rod to pressurize the sheath removal hole model at 130 g/cm² (the pressure corresponding to the artery pressure). The welding laser was then radiated from outside the glass rod. The laser used was a semiconductor laser having a wavelength of 810 nm, and radiated under the condition of 0.37 W/mm² for 8 seconds.

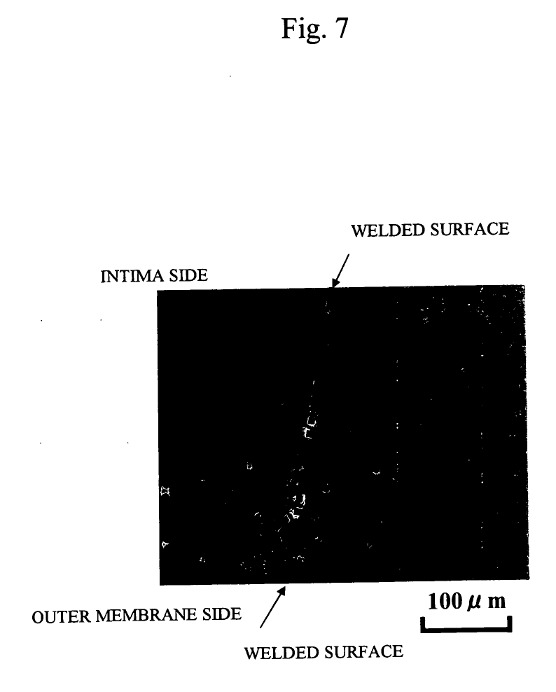

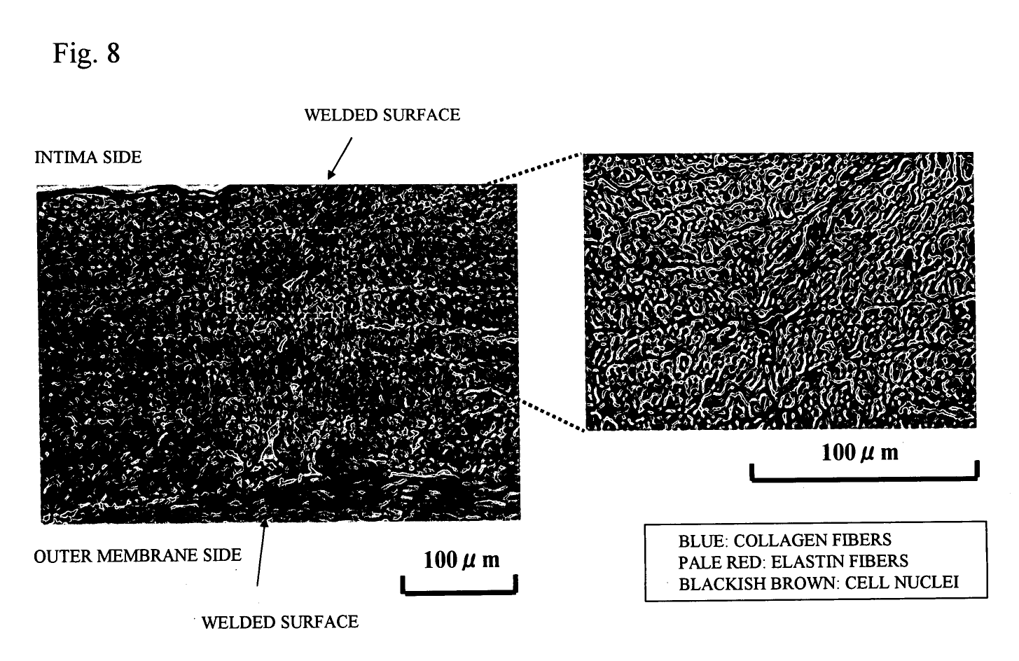

As a result, the entire sheath removal hole was closed by welding. FIG. 7 shows a photograph of a cross section in the welded site. In the photograph of FIG. 7, the upper side shows the intima side of the blood vessel, and the lower side shows the outer membrane side. FIG. 8 is a photograph of the cross section of the welded surface stained with Masson Trichrome (MT) to show the tissue characterization. In this staining, collagen fibers are stained blue, elastin fibers are stained pale red, and cell nuclei are stained blackish brown. It was found from the staining photograph that collagen was entangled and welded.

### EXAMPLE 2

Measurements of backscattered light at the fiber end

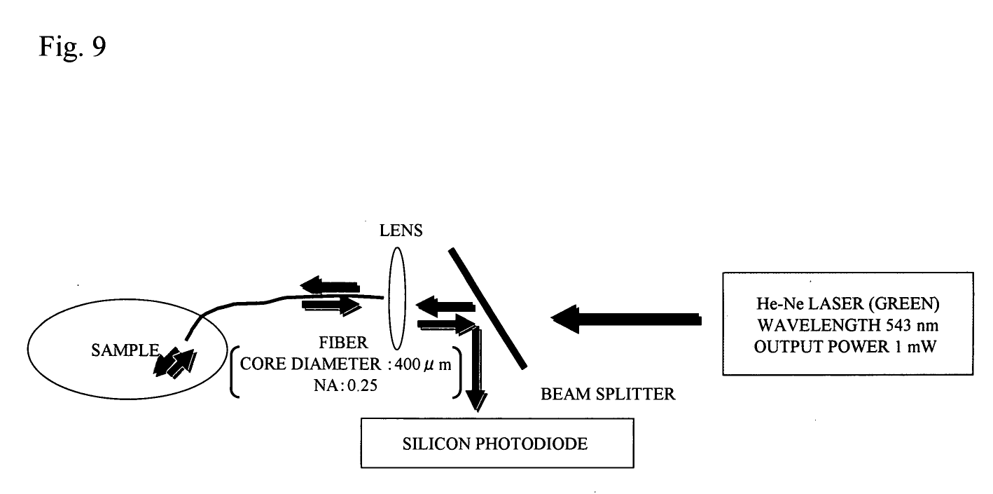

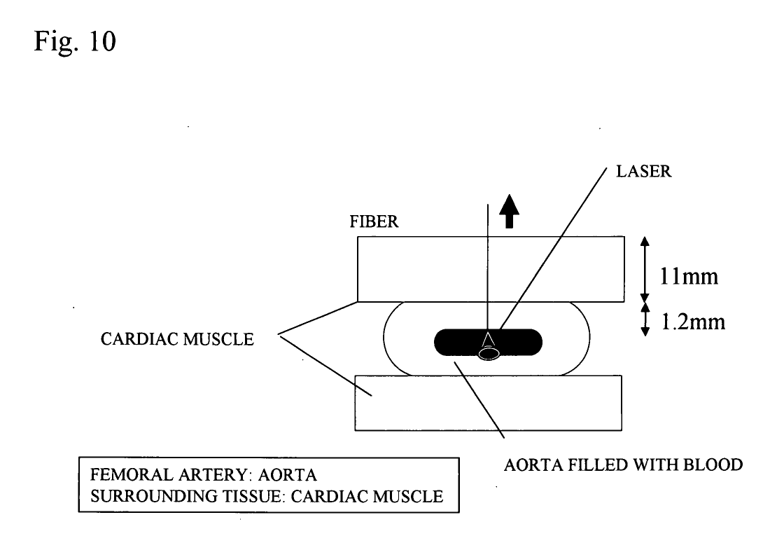

An aorta of a pig was used as a blood vessel and a cardiac muscle of a pig was used as a surrounding tissue to construct a model that simulates a blood vessel and a surrounding tissue, as described below. Two slices of cardiac muscle cut into a thickness of 11 mm were prepared, and the aorta of a pig filled with pig blood was sandwiched between the two slices of cardiac muscle. The thickness of the aorta of a pig was 1.2 mm, and the distance from the center of the blood vessel to the intima of the vessel wall was 0.5 mm (FIG. 10). A quartz fiber (core diameter: 400 μm, NA: 0.25) was connected to a He—Ne laser (543 nm in wavelength, 1 mW in output power) generator (LASOS, Model LGK7786P50). At this time, a lens and a beam splitter were provided between the quartz fiber and the laser generator in this order from the quartz fiber side. The beam splitter was provided so that light arriving at the beam splitter from the fiber side could alter the course, and a silicon photodiode was provided so that the light that altered the course could reach the silicon photodiode (FIG. 9). The end of the fiber was inserted into the model so that the fiber end could penetrate the cardiac muscle and vessel wall and could be located in the blood. While weak light was being radiated from the laser generator, the fiber end was moved from within the blood into the aorta wall into the cardiac muscle, and backscattered light that could be monitored at the silicon photodiode through the fiber was measured over time. Arrows in FIG. 9 show directions of the courses of light. The He—Ne laser generated in the laser generator is directed into the fiber through the lens as shown by gray arrows and proceeds in the fiber to the fiber end. The light is radiated into a sample (the model that simulates a blood vessel and a surrounding tissue), absorbed and scattered, and then returned into the fiber as the backscattered light. In FIG. 9, the courses of the backscattered light are shown by black arrows. The backscattered light alters the course at the beam splitter, and enters a photodetector (silicon photodiode) and measured.

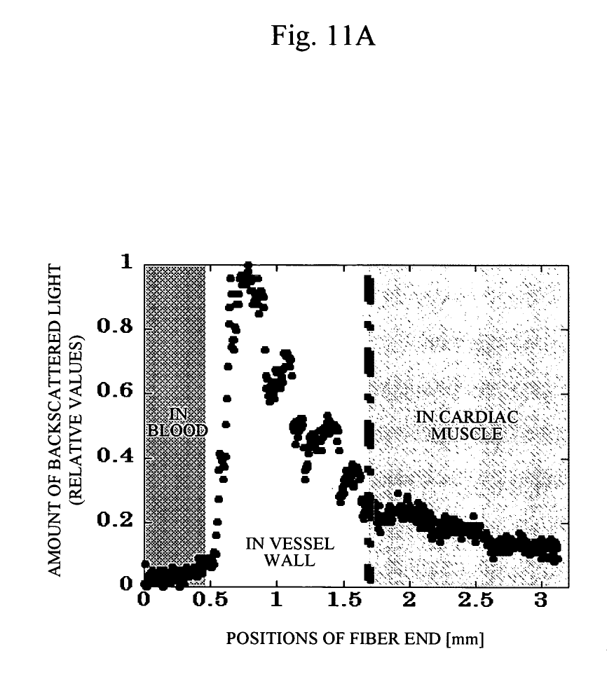

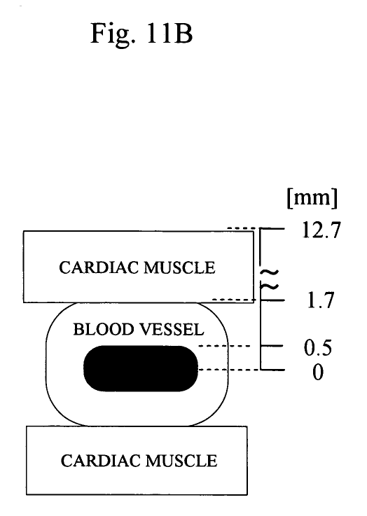

The result is shown in FIG. 11A. As shown in FIG. 11A, the backscattered light was very weak while the fiber end was in the blood. However, it abruptly increased and slowly decreased in the vessel wall, and further decreased in the cardiac muscle. That is to say, in the three-layered model of blood, blood vessel, and cardiac muscle, positions of the fiber end corresponded to the amount of backscattered light from tissues. FIG. 11B shows materials used in the experiment.

### EXAMPLE 3

Evaluation of weld strength with respect to a closed sheath removal hole

The weld strength of the sheath removal hole closed using the method of First Example was evaluated by means of a weld strength evaluating device (lumen pressurizing device).

Structure of the weld strength evaluating device

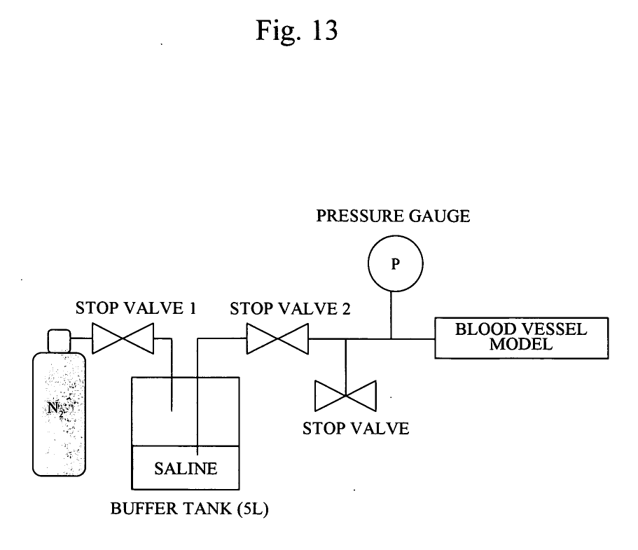

The intravascular lumen pressurizing device is comprised of an nitrogen cylinder (Toyokokagaku Co, Ltd., Kanagawa), a 5 L buffer tank (stainless pressurized container TM5SRV, AS ONE CORPORATION, Tokyo), a stop valve (integral bonnet needle valve B-1RS4, Swagelok Company, OH), a pressure gauge (environmental proof digital pressure sensor AP-13S, Keyence Corporation, Osaka), and a vinyl tube. The buffer tank has a structure that discharges liquid when pressurized by gas. In the experiment, nitrogen is used as the gas and normal saline solution (Otsuka Isotonic sodium chloride solution (registered trademark), Otsuka Pharmaceutical Co., Ltd., Tokyo) is used as the liquid. A luer fitting (VRM206, ISIS Co., Ltd., Osaka) is used to mount the blood vessel model on the end of the vinyl tube. The pressure of the entire system is increased to a pressure corresponding to the artery pressure with valves 1 and 2 open, and thereafter, only the valve 1 is closed and the pressure of the entire system is maintained at a constant level. The pressure gauge may be used to measure the pressure. Because the capacity of the buffer tank (5 L) is sufficiently small with respect to the capacity of the blood vessel model (approximately 50 mL), and therefore the pressure loss due to the leakage of the normal saline solution from the blood vessel model is small, the device allows the pressure of the entire system to be maintained even if the normal saline solution leaks from the blood vessel model. FIG. 13 shows the intravascular lumen pressurizing device that was used.

Weld strength when tunica media welding has been achieved

As a result of lumen pressurizing using normal saline solution applied on a sample in which tunica media welding has been achieved, no leakage of the normal saline solution was evident until the lumen approaches 202 mmHg, and the catheter sheath removal hole had been completely sealed. A closing strength twice the artery pressure in humans (approximately 100 mmHg) was obtained.

### INDUSTRIAL APPLICABILITY

The device according to the invention may be used to radiate weak light onto a sheath removal hole and measure backscattered light of the light, and thereby determine the location of the end of the optical fiber that radiates the light. Then, if it is determined that the position of the end of the optical fiber is in the vessel wall, welding laser may be radiated, causing softened collagen to be entangled to weld and close the sheath removal hole. The device according to the invention may be used to radiate the welding laser only on the vessel wall having the sheath removal hole formed without radiating the laser onto other tissues. As shown in Third Example, the sheath removal hole closed by means of the sheath removal hole closing device according to the invention is reliably closed such that a lumen pressure twice that of the artery blood causes no leakage of liquid.

All publications, patents, and patent applications sited herein are incorporated herein in their entirety by reference.

1. A device for closing a sheath removal hole formed in the wall of a blood vessel by laser welding, comprising welding laser generating means, means for transmitting the welding laser, and means for monitoring the position of an end of the welding laser transmitting means, wherein the welding laser is radiated when the end of the welding laser transmitting means is located in the vessel wall.

2. The device for closing a sheath removal hole according to claim 1, wherein the welding laser is a laser capable of heating the vessel wall.

3. The device for closing a sheath removal hole according to claim 2, wherein the welding laser is a continuous laser capable of heating the vessel wall.

4. The closing device sheath removal hole according to claim 3, wherein the welding laser is selected from the group consisting of a semiconductor laser, a Nd:YAG laser, and a second harmonics of a Nd:YAG laser.

5. The closing device sheath removal hole according to any one of claims 1 to 4, wherein the means for monitoring the position of an end of the welding laser transmitting means includes means for generating monitoring light, means for transmitting the monitoring light, and means for detecting backscattered light of the monitoring light, and wherein an end of the monitoring light transmitting means and the end of the welding laser transmitting means are located at the same position, the monitoring light that is light having a wavelength absorbable by substances present in blood is radiated, the backscattered light of the radiated monitoring light is detected, and the position of the end of the welding laser transmitting means is determined based on the intensity of the detected light.

6. The closing device sheath removal hole according to claim 5, wherein in the means for monitoring the position of an end of the welding laser transmitting means, the monitoring light is light having a wavelength absorbable by hemoglobin, and wherein it can be determined where the end of the welding laser transmitting means is located; in blood, in the vessel wall, or in a surrounding tissue of the blood vessel.

7. The closing device sheath removal hole according to claim 6, the light having a wavelength absorbable by hemoglobin used to monitor the position of an end of the welding laser transmitting means is selected from the group consisting of a semiconductor laser having a wavelength of 810 nm, a He—Ne laser having a wavelength of 543 nm, and a second harmonics of Nd:YAG laser having a wavelength of 532 nm.

8. The closing device sheath removal hole according to any one of claims 1 to 7, wherein the welding laser transmitting means and the monitoring light transmitting means are a common flexible transmitting means.

9. The closing device sheath removal hole according to claim 8, wherein the flexible transmitting means is selected from the group consisting of a quartz glass fiber, a plastic fiber, and a hollow medical waveguide.

10. The closing device sheath removal hole according to any one of claims 1 to 9, wherein the welding laser generating means and the monitoring light generating means are a common semiconductor laser or Nd:YAG laser second harmonics generator.

11. The sheath removal hole closing device according to any one of claims 1 to 10, further comprising a means for supplying a welding laser energy absorbing pigment to the sheath removal hole.

12. The closing device sheath removal hole according to claim 11, wherein the welding laser energy absorbing pigment is indocyanine green.

13. A control method for determining the position of an end of a light transmitting fiber and radiating a welding laser through the light transmitting fiber onto the wall of a blood vessel in which a sheath removal hole is formed, in order to close the sheath removal hole by laser welding, comprising the steps of:
(a) radiating weak light used for determining a surrounding tissue through the light transmitting fiber inserted in the sheath inserted in the blood vessel, the fiber connected with a light generator;
(b) measuring backscattered light of the radiated weak light by a detector;
(c) determining a tissue surrounding the end of the light transmitting fiber; and
(d) radiating welding laser if it is determined that the tissue surrounding the end of the light transmitting fiber is the vessel wall.

14. A device for monitoring the position of an end of monitoring light transmitting means, comprising means for generating monitoring light, means for transmitting the monitoring light, and means for detecting backscattered light of the monitoring light, wherein the monitoring light that is light having a wavelength absorbable by substances present in blood is radiated, the backscattered light of the radiated monitoring light is detected, and the position of the end of the monitoring light transmitting means is determined based on the intensity of the detected light.

15. The device for monitoring the position of an end of a monitoring light transmitting means according to claim 14, wherein in means for monitoring the position of an end of the monitoring light transmitting means, the monitoring light is light having a wavelength absorbable by hemoglobin, and wherein it can be determined where the end of the monitoring light transmitting means is located; in blood, in the vessel wall, or in a surrounding tissue of the blood vessel

16. The device for monitoring the position of an end of a monitoring light transmitting means according to claim 14 or 15, the light having a wavelength absorbable by hemoglobin used to monitor the position of an end of the monitoring light transmitting means is selected from the group consisting of a semiconductor laser having a wavelength of 810 nm, a He—Ne laser having a wavelength of 543 nm, and a second harmonics of Nd:YAG laser having a wavelength of 532 nm.
